(19) United States
(12) Patent Application Publication
(10) Pub. No.: US 2026/0207091 A1
(43) Pub. Date: Jul. 23, 2026

(54) SINGLE SOURCE-DETECTOR SEPARATION APPROACH TO CALCULATE TISSUE OXYGEN SATURATION

(71) Applicant: The United States of America, as represented by the Secretary, Department of Health and Human Servic, Bethesda, MD (US)

(72) Inventors: Thien Nguyen, McLean, VA (US); Amir Hossein Gandjbakhche, Potomac, MD (US); Soongho Park, McLean, VA (US); Brian Y. Hill, Washington, DC (US)

(73) Assignee: The United States of America, as represented by the Secretary, Department of Health and Human Servic, Bethesda, MD (US)

(21) Appl. No.: 19/140,070

(22) PCT Filed: Dec. 22, 2023
(86) PCT No.: PCT/US2023/085725

§ 371 Date: Jun. 17, 2025

Related U.S. Application Data
(60) Provisional application No. 63/434,827, filed on Dec. 22, 2022.

(51) Int. Cl. A61B 5/1455; A61B 5/0205; A61B 5/024; G16H 40/67

(52) U.S. Cl. CPC A61B 5/14552; A61B 5/02055; G16H 40/67; A61B 5/02416; A61B 2562/0238; A61B 2562/0242; A61B 2562/164

(57) ABSTRACT

A tissue oxygen saturation (StO₂) parameter is calculated using a single light source and single detector of an optical device, where the light source and detector are separated by a separation distance. A single source-detector separation (SSDS) approach to measure StO₂ in the tissue of a subject utilizes only intensity measurements of three different wavelengths of light emitted from the same light source (or one or more wavelengths from adjacent one or more light sources) at the detector. In addition to the three intensity measurements, the other inputs to the calculation are a differential pathlength factor (DPF) and extinction coefficients for both oxy-hemoglobin and deoxy-hemoglobin for the given wavelengths. The StO₂ parameter can be calculated for each distinct source-detector pair of an optical device.

### CROSS-REFERENCE TO RELATED APPLICATIONS

This patent application is the U.S. national phase of International Application No. PCT/US2023/085725, filed on Dec. 22, 2023, which claims the benefit of U.S. Provisional Patent Application No. 63/434,827, filed Dec. 22, 2022, the disclosures of which are incorporated by reference herein in their entireties for all purposes.

### STATEMENT REGARDING FEDERALLY SPONSORED RESEARCH OR DEVELOPMENT

This invention was made with Government support under Project Number ZO1-HD000261 awarded by the National Institutes of Health. The Government has certain rights in this invention.

### BACKGROUND

Many medical devices are designed to monitor one or more biological parameters related to a patient's health, such as a blood oxygen level, heart rate, or the like. For example, hospital staff may carry a mobile pulse oximeter that clips over a patient's finger and uses a light source to measure the oxygen saturation level of a user's red blood cells. Recently, common consumer devices such as smart watches or fitness bands have incorporated IR emitters and detectors therein in order to perform pulse oximetry measurements. Furthermore, more advanced medical devices may incorporate multiple light sources and multiple detectors to perform more advanced measurements than the conventional single-source, single-detector devices. Increasing the number of sources and/or detectors, thereby having multiple source-detector separation distances implemented on a single device, can enable the capture of additional information that provides insights into more complex parameters than traditional pulse oximetry.

Tissue oxygen saturation (StO₂) is one such important parameter used to assess oxygen delivery and uptake in tissue of a patient. Hypoxia, a term used to indicate inadequate StO₂, is often seen in patients with cardiac problems, respiratory infections, or pulmonary diseases. Prolonged hypoxia can damage vital organs such as the brain, lungs, and heart, and can tragically lead to death. Due to its critical role in maintaining organ function, StO₂ has been frequently used to monitor a patient's health. Common sites used to measure StO₂ include the brain, spinal cord, peripheral muscles, legs, kidneys, and placenta.

One conventional technique to non-invasively measure a tissue oxygen saturation (StO₂) parameter uses near-infrared spectroscopy (NIRS). There are three main types of NIRS: time domain (TD), frequency domain (FD), and continuous wave (CW) NIRS. While TD- and FD-NIRS can provide absolute hemoglobin concentration, these systems are often complex, bulky, and expensive. CW-NIRS-based systems, such as systems employing diffuse reflectance spectroscopy (DRS) and/or spatially resolved spectroscopy (SRS), can be made simpler, wearable, and affordable, but they still require at least two source-detector separations (i.e., at least two different distances between one or more light sources and one or more detectors).

Requiring the use of multiple source-detector separations increases the cost of these devices. Furthermore, including multiple light sources and/or detectors can increase the number of components of the device that may fail or provide erroneous data, thereby leading to incorrect measurements or the inability to conduct a measurement at all. Requiring multiple source-detector separation distances also sets a lower limit as to the size of the measuring device, thereby hindering the miniaturization of the technology. Thus, there is a need for addressing these issues and/or other issues associated with the prior art.

### SUMMARY

Embodiments of the present disclosure relate to techniques for calculating tissue oxygen saturation using a single light source and single detector separated by a separation distance. A single source-detector separation (SSDS) approach to measure StO₂ in a subject is disclosed. The advantage of the SSDS approach lies in its simplicity thanks to the need for only one source-detector pair, which will greatly benefit miniaturized technologies and advance current NIRS technologies by providing more information without changing hardware design. The simple hardware design can also reduce cost and/or size of current wearable devices that implement NIRS technology.

In accordance with a first aspect of the present disclosure, an optical device for measuring a tissue oxygen saturation parameter is provided. The optical device includes: a light source that generates light having at least three different wavelengths; and a first photodetector, separated from the light source by a first separation distance, to sample an intensity of light received from the light source at the first photodetector for each of the at least three different wavelengths. The intensity of light for each of the at least three different wavelengths is used to calculate a first parameter that indicates a tissue oxygen saturation level in one or more layers of tissue between the light source and the first photodetector, and the first parameter is calculated without the input of any intensity of light measured by any photodetector located at a different separation distance from the light source.

In accordance with at least one embodiment of the first aspect, the optical device further includes: a flexible substrate; and logic comprising at least a transceiver connected to an antenna to implement a wireless interface.

In accordance with at least one embodiment of the first aspect, the logic is configured to transmit sample data to at least one of a mobile device or a remote computing device to calculate the first parameter.

In accordance with at least one embodiment of the first aspect, the logic further comprises a processor and a memory, and the processor calculates the first parameter locally and transmits the first parameter to at least one of a mobile device or a remote computing device.

In accordance with at least one embodiment of the first aspect, the optical device further includes at least one of: an inertial measurement unit (IMU) to capture motion data; or a temperature sensor to capture temperature data.

In accordance with at least one embodiment of the first aspect, at least one of the motion data or the temperature data are transmitted to at least one of a mobile device or a remote computing device via the wireless interface.

In accordance with at least one embodiment of the first aspect, the parameter is calculated according to the following equation:

$${StO}_{2} = \frac{\begin{matrix}D P {F}_{3} \cdot log ( {k}_{1} \cdot {I}_{{λ}_{2}} / {I}_{{λ}_{1}} ) \cdot {ε}_{{λ}_{2}}^{Hb} - D P {F}_{1} \cdot log ( {k}_{2} \cdot {I}_{{λ}_{2}} / {I}_{{λ}_{3}} ) \cdot {ε}_{{λ}_{1}}^{Hb} + \\ D P {F}_{2} \cdot log ( {k}_{2} \cdot {I}_{{λ}_{1}} / {k}_{1} \cdot {I}_{{λ}_{3}} ) \cdot {ε}_{{λ}_{2}}^{Hb}\end{matrix}}{\begin{matrix}D P {F}_{1} \cdot log ( {k}_{2} \cdot {I}_{{λ}_{2}} / {I}_{{λ}_{3}} ) ( {ε}_{{λ}_{1}}^{HbO} - {ε}_{{λ}_{1}}^{Hb} ) - D P {F}_{3} \cdot log ( {k}_{1} \cdot {I}_{{λ}_{2}} / {I}_{{λ}_{1}} ) \\ ( {ε}_{{λ}_{3}}^{HbO} - {ε}_{{λ}_{3}}^{Hb} ) + D P {F}_{2} \cdot log ( {k}_{1} \cdot {I}_{{λ}_{3}} / {k}_{2} \cdot {I}_{{λ}_{1}} ) ( {ε}_{2}^{HbO} - {ε}_{{λ}_{2}}^{Hb} )\end{matrix}} ,$$

where DPFᵢ is a differential pathlength factor, kᵢ is a ratio of intensity of light emitted by the light source for two wavelengths, I_{λi} is the measured intensity of light at the photodetector,

$${ε}_{{λ}_{i}}^{Hb}$$

is the extinction coefficient for deoxy-hemoglobin,

$${ε}_{{λ}_{i}}^{HbO}$$

is the extinction coefficient for oxy-hemoglobin, and i is an index for the different wavelengths of light.

In accordance with at least one embodiment of the first aspect, the DPFᵢ is constant for each of the three wavelengths.

In accordance with at least one embodiment of the first aspect, the DPFᵢ is calculated for each wavelength according to the following equation:

$$D P {F}_{i} = \frac{1}{2} {( \frac{3 {μ}_{s}^{′} ( {λ}_{i} )}{{μ}_{a} ( {λ}_{i} )} )}^{1 / 2} ,$$

where

$${μ}_{s}^{′} = 1 {mm}^{- 1}$$

and μₐ is an absorption coefficient.

In accordance with at least one embodiment of the first aspect, the optical device further includes: a second photodetector, separated from the light source by a second separation distance, to sample an intensity of light received from the light source at the second photodetector for each of the at least three different wavelengths. The intensity of light for each of the at least three different wavelengths is used to calculate a second parameter that indicates a tissue oxygen saturation level in one or more layers of tissue between the light source and the second photodetector, and the second parameter is calculated without the input of any intensity of light measured by the first photodetector located at the first separation distance from the light source.

In accordance with at least one embodiment of the first aspect, the optical device further includes one or more additional photodetectors to measure a second parameter. The second parameter can be tissue oxygen saturation or any other biological signal such a heart rate, pulse oxidation, blood pressure, or the like.

In accordance with at least one embodiment of the first aspect, a second light source, separated from the photodetector by a second separation distance, to sample an intensity of light received from the second light source at the photodetector for each of the at least three different wavelengths. The intensity of light for each of the at least three different wavelengths is used to calculate a second parameter that indicates a tissue oxygen saturation level in one or more layers of tissue between the second light source and the photodetector, and the second parameter is calculated without the input of any intensity of light measured by the photodetector located at the first separation distance from the first light source.

In accordance with at least one embodiment of the first aspect, the optical device further includes one or more additional light sources of one or more wavelengths to measure a second parameters. The second parameter can be a measurement of tissue oxygen saturation or any other biological signal such a heart rate, pulse oxidation, or the like.

In accordance with at least one embodiment of the first aspect, the optical device further includes: at least one additional light source; and a plurality of additional photodetectors arranged in a two- or three-dimensional pattern. For each additional photodetector of the plurality of additional photodetectors, the intensity of light for each of the at least three different wavelengths is used to calculate a corresponding parameter that indicates a tissue oxygen saturation level in one or more layers of tissue between a particular light source and the additional photodetector, the particular light source is selected from the light source or one of the at least one additional light source, and the corresponding parameter is calculated without the input of any intensity of light measured by any of the other additional photodetectors located at a different separation distance from the particular light source. A plurality of corresponding parameters for the plurality of additional photodetectors are used to construct a parameter map.

In accordance with at least one embodiment of the first aspect, the optical device further includes one or more additional light sources of one or more wavelengths and one or more additional photodetectors to measure a second parameter. The second parameter can be a measurement of tissue oxygen saturation or any other biological signal such a heart rate, pulse oxidation, or the like.

In accordance with at least one embodiment of the first aspect, the one or more additional light sources and one or more additional photodetectors can be located on the same surface or opposite surfaces of a substrate. In accordance with at least one embodiment of the first aspect, the one or more additional light sources and one or more additional photodetectors are arranged in a three-dimensional pattern on two or more surfaces of a substrate.

In accordance with at least one embodiment of the first aspect, the light source includes multiple light sources with at least one wavelength, wherein each of the multiple light sources are placed close to the other multiple light sources to act as a multi-wavelength light source.

In accordance with at least one embodiment of the first aspect, the optical device further includes: a network interface controller (NIC) configured to connect to a network, wherein the optical device transmits a message to a server device via the network, the message including at least one parameter that indicates a tissue oxygen saturation level measured by the optical device.

In accordance with a second aspect of the present disclosure, a system for determining a tissue oxygen saturation StO₂ parameter of an in-vivo tissue of a subject is provided. The system includes: an optical device; and a remote computing device in communication with the optical device. At least one of the optical device or the remote computing device is configured to calculate the StO₂ parameter in accordance with a single source-detector separation (SSDS)-based method that is based on light intensity measurements performed by the optical device for at least three wavelengths of light emitted by a light source and measured at a first photodetector separated from the light source by a first separation distance.

In accordance with at least one embodiment of the second aspect, the StO₂ parameter is calculated according to the following equation:

$${StO}_{2} = \frac{\begin{matrix}D P {F}_{3} \cdot log ( {k}_{1} \cdot {I}_{{λ}_{2}} / {I}_{{λ}_{1}} ) \cdot {ε}_{{λ}_{2}}^{Hb} - D P {F}_{1} \cdot log ( {k}_{2} \cdot {I}_{{λ}_{2}} / {I}_{{λ}_{3}} ) \cdot {ε}_{{λ}_{1}}^{Hb} + \\ D P {F}_{2} \cdot log ( {k}_{2} \cdot {I}_{{λ}_{1}} / {k}_{1} \cdot {I}_{{λ}_{3}} ) \cdot {ε}_{{λ}_{2}}^{Hb}\end{matrix}}{\begin{matrix}D P {F}_{1} \cdot log ( {k}_{2} \cdot {I}_{{λ}_{2}} / {I}_{{λ}_{3}} ) ( {ε}_{{λ}_{1}}^{HbO} - {ε}_{{λ}_{1}}^{Hb} ) - D P {F}_{3} \cdot log ( {k}_{1} \cdot {I}_{{λ}_{2}} / {I}_{{λ}_{1}} ) \\ ( {ε}_{{λ}_{3}}^{HbO} - {ε}_{{λ}_{3}}^{Hb} ) + D P {F}_{2} \cdot log ( {k}_{1} \cdot {I}_{{λ}_{3}} / {k}_{2} \cdot {I}_{{λ}_{1}} ) ( {ε}_{2}^{HbO} - {ε}_{{λ}_{2}}^{Hb} )\end{matrix}} ,$$

where DPFᵢ is a differential pathlength factor, kᵢ is a ratio of intensity of light emitted by the light source for two wavelengths, I_{λi} is the measured intensity of light at the photodetector,

$${ε}_{{λ}_{i}}^{Hb}$$

is the extinction coefficient for deoxy-hemoglobin

$${ε}_{{λ}_{i}}^{HbO}$$

is the extinction coefficient for oxy-hemoglobin, and i is an index for the different wavelengths of light.

In accordance with at least one embodiment of the second aspect, the remote computing device comprises one of a mobile device, a tablet computer, a laptop computer, a personal computer, or a server computer, and the remote computing device comprises at least one processor and a memory, the at least one processor configured to calculate the StO₂ parameter.

In accordance with at least one embodiment of the second aspect, the remote computing device is configured to execute a machine learning algorithm based on at least one StO₂ parameter measured by the optical device.

In accordance with at least one embodiment of the second aspect, the optical device comprises at least two photodetectors, and each photodetector of the at least two photodetectors is used to calculate a corresponding StO₂ parameter for a different location in the in-vivo tissue.

In accordance with at least one embodiment of the second aspect, the at least two photodetectors are arranged in a two- or three-dimensional pattern, and the optical device further includes a plurality of light sources.

In accordance with at least one embodiment of the second aspect, the optical device further includes at least one auxiliary sensor for measuring at least one of: a relative change in oxy-hemoglobin or deoxy-hemoglobin; a blood oxygen saturation (SpO₂); heart rate; heart rate variability; breathing rate; breathing depth; temperature; or motion.

In accordance with a second aspect of the present disclosure, a method for determining a tissue oxygen saturation StO₂ parameter is provided. The method includes: measuring, using a first photodetector of an optical device, an intensity of light for at least three wavelengths of light emitted by a light source to generate sample data, wherein the light source is positioned a first separation distance from the first photodetector; and calculating a StO₂ parameter using a single source-detector separation (SSDS)-based method in accordance with the following equation:

$${StO}_{2} = \frac{\begin{matrix}D P {F}_{3} \cdot log ( {k}_{1} \cdot {I}_{{λ}_{2}} / {I}_{{λ}_{1}} ) \cdot {ε}_{{λ}_{2}}^{Hb} - D P {F}_{1} \cdot log ( {k}_{2} \cdot {I}_{{λ}_{2}} / {I}_{{λ}_{3}} ) \cdot {ε}_{{λ}_{1}}^{Hb} + \\ D P {F}_{2} \cdot log ( {k}_{2} \cdot {I}_{{λ}_{1}} / {k}_{1} \cdot {I}_{{λ}_{3}} ) \cdot {ε}_{{λ}_{2}}^{Hb}\end{matrix}}{\begin{matrix}D P {F}_{1} \cdot log ( {k}_{2} \cdot {I}_{{λ}_{2}} / {I}_{{λ}_{3}} ) ( {ε}_{{λ}_{1}}^{HbO} - {ε}_{{λ}_{1}}^{Hb} ) - D P {F}_{3} \cdot log ( {k}_{1} \cdot {I}_{{λ}_{2}} / {I}_{{λ}_{1}} ) \\ ( {ε}_{{λ}_{3}}^{HbO} - {ε}_{{λ}_{3}}^{Hb} ) + D P {F}_{2} \cdot log ( {k}_{1} \cdot {I}_{{λ}_{3}} / {k}_{2} \cdot {I}_{{λ}_{1}} ) ( {ε}_{2}^{HbO} - {ε}_{{λ}_{2}}^{Hb} )\end{matrix}} ,$$

where DPFᵢ is a differential pathlength factor, kᵢ is a ratio of intensity of light emitted by the light source for two wavelengths, I_{λi} is the measured intensity of light at the photodetector,

$${ε}_{{λ}_{i}}^{Hb}$$

is the extinction coefficient for deoxy-hemoglobin,

$${ε}_{{λ}_{i}}^{HbO}$$

is the extinction coefficient for oxy-hemoglobin, and i is an index for the different wavelengths of light.

### BRIEF DESCRIPTION OF THE DRAWINGS

The present systems and methods for calculating a tissue oxygen saturation parameter are described in detail below with reference to the attached drawing figures, wherein:

FIG. 1A illustrates an optical device, in accordance with some embodiments;

FIG. 1B illustrates an optical device, in accordance with some other embodiments;

FIG. 2 illustrates a system including an optical device and a remote computing device, in accordance with some embodiments;

FIG. 3 illustrates the conceptual operation of the device, in accordance with some embodiments;

FIG. 4 illustrates an optical device, in accordance with yet other embodiments;

FIG. 5 is a flowchart of a method for measuring a parameter using the device, in accordance with some embodiments;

FIG. 6 illustrates a comparison between actual and calculated StO₂ parameters in an optical simulation when DPF values are specified, in accordance with some embodiments;

FIG. 7 illustrates a cerebral oxygen saturation calculated using a conventional spatially resolved spectroscopy (SRS) method and the proposed single source-detector separation (SSDS) method, in accordance with some embodiments;

FIG. 8 illustrates a difference in calculated StO₂ parameters compared between the SRS and the SSDS methods, in accordance with some embodiments;

FIG. 9 illustrates an exemplary computer system, in accordance with some embodiments;

FIG. 10 illustrates an example use case of an optical device, in accordance with some embodiments;

FIG. 11 shows the time-series data sampled using the device shown in FIG. 10, in accordance with some embodiments; and

FIG. 12 illustrates a map of cerebral tissue oxygen saturation levels, in accordance with some embodiments.

### DETAILED DESCRIPTION

Systems and methods are disclosed for measuring StO₂ in a subject using a single source-detector separation (SSDS) distance. The SSDS approach described herein was developed based on a relationship between the attenuation difference of a selected wavelength pair and a proportion of oxy-hemoglobin and met-myoglobin in meat tissue. The main absorbance of meat tissue is myoglobin, whereas the dominant absorbance of in-vivo human tissues is hemoglobin. However, hemoglobin has a similar spectral extinction with myoglobin. Therefore, it is possible to use the attenuation differences from certain wavelength pairs to compute the proportion of oxy-hemoglobin (HbO) in an in-vivo human tissue, which is equivalent to the measured StO₂ parameter. The advantage of this approach is that it only requires a single source-detector pair separated by a single separation distance in order to calculate the StO₂ parameter. Compared to a conventional technique that requires multiple separation distances, this can reduce the complexity of a device by requiring fewer components.

The devices described herein can be incorporated into consumer devices such as wearable activity trackers, smart watches, or the like in order to track health parameters like StO₂. Alternatively, the devices can be incorporated into a commercial product intended for clinical use, such as a device for monitoring patients in a hospital setting or doctor's office.

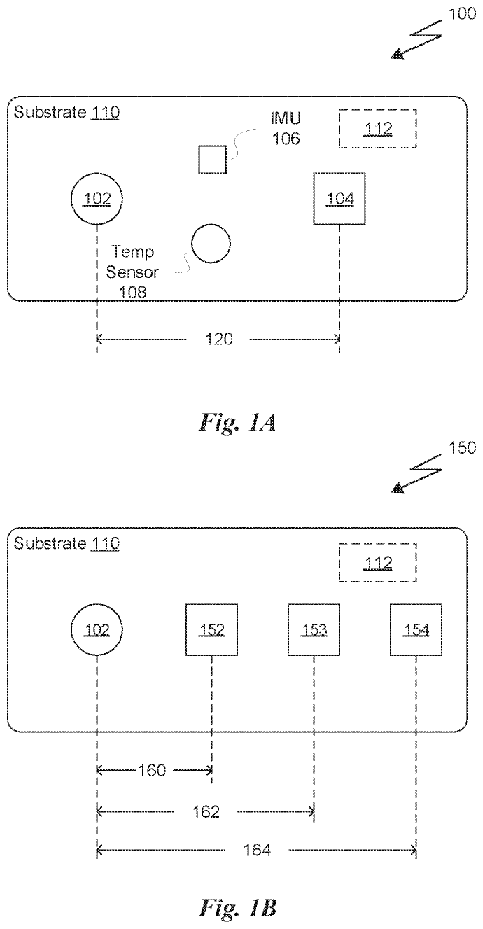

FIG. 1A illustrates an optical device 100, in accordance with some embodiments. The device 100 includes at least one multi-wavelength light source 102 and at least one photodetector 104, to non-invasively measure in-vivo StO₂ in a subject (e.g., in a tissue of the subject placed proximate the device 100). The device 100 can be applied to measure StO₂ in different tissues such as muscle tissue, brain tissue, spinal cord tissue, internal organ tissues, fetal tissue, and placental tissue. In an embodiment, the light source 102 is a multi-wavelength LED diode (e.g., includes multiple LED diodes of different wavelengths), and the photodetector 104 can be at least one of a photodiode, a photodiode array, an avalanche photodiode, or a photomultiplier tube. In another embodiment, the light source 102 can be a laser or laser diode. The light source 102 can also be directed by a number of optical components such as one or more lenses, optical media, waveguides, and/or optical fibers. It will be appreciated that a multi-wavelength light source can refer to a group of multiple light sources with at least one wavelength, wherein each of the multiple light sources are placed close to the other multiple light sources to act as a multi-wavelength light source. In another embodiment, multiple light sources of at least one wavelength can be located at different locations, but light output from each light source may be directed, e.g., through optical fibers, waveguides, or the like, to a same location.

In an embodiment, the device 100 can also include other auxiliary sensors such as, but not limited to, an inertial measurement unit (IMU) 106 and a temperature sensor 108. The IMU 106 can also be referred to as a motion sensor, and can include one or more gyroscopes and/or accelerometers for tracking a position and/or orientation of the device 100. For example, when the device 100 is worn by a patient, the IMU 106 can be used to track the movement of the patient over time, thus measuring steps, altitude, or the like, and can be used to correlate the patient's motion with the various monitored health parameters such as StO₂.

The light source 102, photodetector 104, and auxiliary sensors 106/108 may be connected to a substrate 110. In an embodiment, the substrate 110 is a flexible substrate. The substrate 110 can be a flexible printed circuit board (PCB) having a dielectric layer such as polyimide and one or more conductive layers of, e.g., copper, with a number of circuits printed (e.g., etched) thereon. It will be appreciated that, in other embodiments, the dielectric material can be selected from any number of other types of polymers and/or elastomers that may have the desired properties for the given application.

In an embodiment, the device 100 may also include logic 112 that may include, but is not limited to, a processor, memory, wireless transceiver, and/or antenna. The logic 112 may be incorporated into a single chip (e.g., a package) soldered to a back side of the substrate 110. In some embodiments, the logic 112 may be embedded in the substrate, such as through an over-molding process. In some embodiments, the logic 112 may be implemented as a chipset including multiple discrete chips. For example, the processor and memory may be included in separate chips, connected through the circuits printed on the surface of the substrate 110.

The device 100 can be configured to measure parameters such as StO₂, body temperature, heart rate, and the like using the sensors included thereon. Signals from the photodetector 104, IMU 106, and/or temperature sensor 108 can be sampled (e.g., using one or more analog-to-digital converters (ADC) included in logic 112) periodically and the sampled values can be stored in the memory. In some embodiments, the device 100 is connected to a remote computing device through a wireless communications interface implemented using the transceiver and antenna. The wireless interface can be near field communication (NFC), Bluetooth, Wi-Fi (IEEE 802.11), or the like. The sampled data can be streamed to the remote computing device in real-time, and the remote computing device stores the sampled data in a memory of the remote computing device. In other embodiments, the device 100 may be configured to log data locally, which may then later be downloaded from the memory through a wired (e.g., USB) or wireless connection.

A parameter like StO₂ is measured by detecting light of different wavelengths emitted by the light source 102 using the photodetector 104. The light source 102 and the photodetector 104 are separated by a fixed separation distance 120. When placed next to the skin or tissue of a subject, the light emitted by the light source 102 penetrates the tissue and is reflected back towards the photodetector 104. The intensity of the light striking the photodetector 104 will generate an electrical signal that can be sampled by the photodetector and converted into a value that indicates the intensity of a particular wavelength of light that reaches the photodetector 104. Typically, light penetrates the tissue up to a depth of approximately half the separation distance 120. Thus, choice of the separation distance 120 is critical to measure StO₂ for a particular tissue layer. More details about the manner of measuring StO₂ will be set forth below.

In an embodiment, the light generated by the light source 102 can be infrared light between approximately 700 nm and 2500 nm wavelengths, such as 730 nm, 800 nm, and/or 850 nm wavelengths in the near infrared spectrum. In other embodiments, the light generated by the light source 102 can be in the visible spectrum, e.g., between 380 and 700 nm wavelengths.

FIG. 1B illustrates an optical device 150, in accordance with some other embodiments. The device 150 differs from the device 100 in that the device 150 includes a plurality of photodetectors 152, 154, 156, separated from the light source 102 by separation distances 160, 162, and 164, respectively. The light source 102 and photodetectors 152, 154, 156 can be arranged to measure StO₂ of multiple tissue layers in one or more locations, based on the arrangement of the light source 102 and photodetectors 152, 154, 156 on the substrate 110, with each measurement calculated based on light detected by a single pair of light source 102 and corresponding photodetector.

Although not shown explicitly in FIG. 1B, the device 150 may also include auxiliary sensors such as the IMU 106 and/or temperature sensor 108. The device 150 may also include logic 112 connected to the substrate 110, details of which are similar to that described above for device 100. Additional sensors, including the IMU 106, temperature sensor 108, and/or additional photodetectors, can be used to measure other parameters including a separate StO₂ parameter for a different layer of tissue or other parameters for other biological signals such as heart rate, pulse oxidation, body temperature, electrocardiogram, or the like.

In various embodiments, the devices 100, 150 can be implemented as: (1) a miniaturized device to be used as parts of wearable and/or sportswear devices; (2) a device that includes multiple light sources and detectors to separate StO₂ of a layer in a multiple layer tissue or to measure StO₂ at multiple locations; or (3) embedded in existing NIRS technologies to provide additional information of StO₂.

In various embodiments, the techniques disclosed herein may be implemented by one or more processors, either included in the wearable device (e.g., as an application specific integrated circuit (ASIC), embedded microcontroller, or RISC-based CPU, for example) or included in a connected device, such as a mobile phone, tablet device, personal computer, server computer, or the like, which may be connected to the wearable device via a wired or wireless communications interface. The one or more processors can implement the various methods or techniques via hardware, software, firmware, or any combination thereof.

For example, the device 150 may sample a light intensity measured by each photodetector 152, 154, 156 for one or more wavelengths of light at a number of sampling times and transmit the digital intensity values to an application executing on a mobile device wirelessly connected to the device 150 for further processing. The application then processes the intensity values to determine the StO₂ parameter for one or more tissue locations.

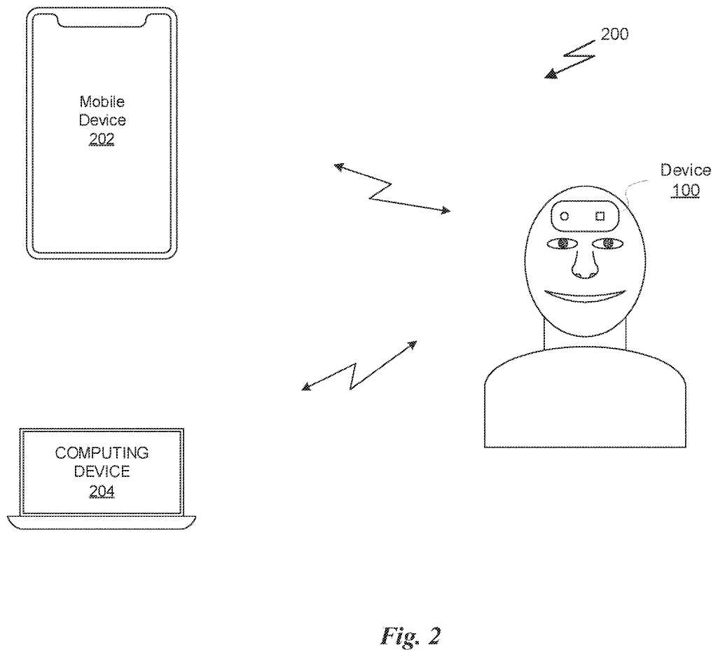

FIG. 2 illustrates a system including an optical device and a remote computing device, in accordance with some embodiments. As shown in FIG. 2, the device 100 can be wirelessly connected to a mobile device 202 and/or a remote computing device 204 using the transceiver and antenna included in logic 112. The mobile device 202 can include, but is not limited to, a cellular phone, tablet computer, or the like. The computing device 204 can include a laptop computer, personal computer, server computer, or the like. The device 100 can communicate with the mobile device 202 and/or the computing device 204 via a direct connection or an indirect connection through, e.g., one or more network devices such as an access point (AP), base station (e.g., eNodeB), switch, router, server devices, or the like.

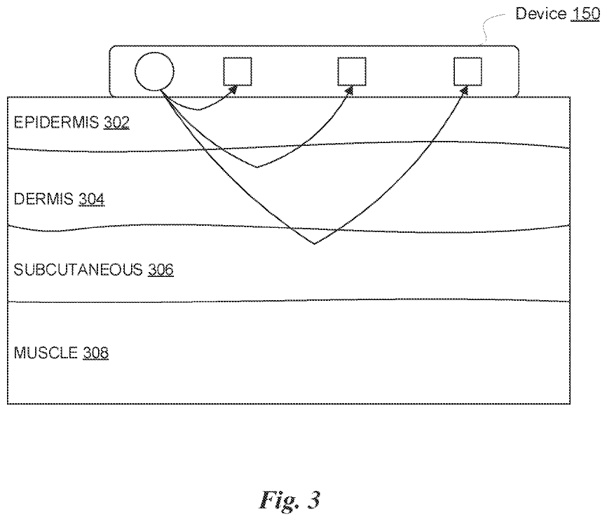

FIG. 3 illustrates the conceptual operation of the device 150, in accordance with some embodiments. As shown in FIG. 3, an optical device 150 is placed on a subject's skin in a location for measuring a StO₂ parameter in one or more layers of tissue. The layers of tissue can include, but are not limited to, an epidermis 302, a dermis 304, a subcutaneous 306, and muscle 308. Additional layers of tissue can include organs (e.g., kidneys, uterus, etc.), fat, fluids, and the like.

It will be appreciated that light from the light source 102 enters the tissue from a top layer and is diffused and/or reflected back towards the three photodetectors 152, 154, and 156. Light tends to penetrate one or more layers of tissue up to about half of the separation distance between a particular light source 102 and a corresponding photodetector. Thus, photodetector 152, having the shortest separation distance 160 from light source 102, may measure light intensity values corresponding to a first layer of tissue (e.g., epidermis 302); photodetector 154, having an intermediate separation distance 162 from light source 102, may measure light intensity values corresponding to a second layer of tissue (e.g., dermis 304); and photodetector 156, having a longest separation distance 164 from light source 102, may measure light intensity values corresponding to a third layer of tissue (e.g., subcutaneous 306). Given the fixed separation distances 160, 162, 164, light may not penetrate far enough to reach the muscle layer 308. However, by implementing different devices with different separation distances between the light source 102 and the photodetectors, designers may create a device to target particular layers at an expected depth for, e.g., measuring the StO₂ parameter in different locations of the body (e.g., brain, placenta, muscles, etc.).

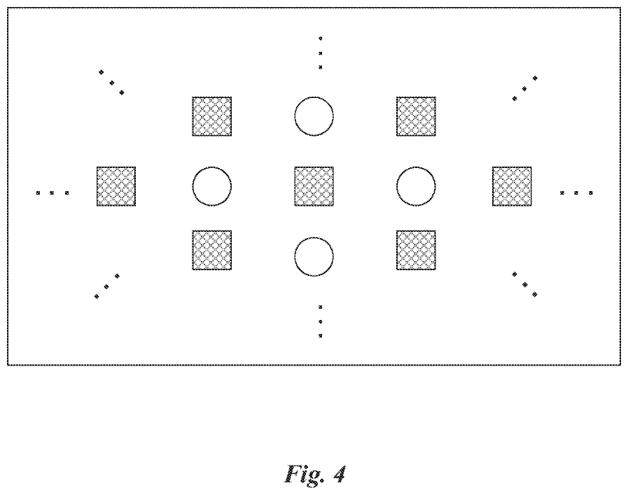

FIG. 4 illustrates an optical device 400, in accordance with yet other embodiments. Some optical devices, such as the device 400, can be designed to have a plurality of light sources (shown as circular elements in FIG. 4) and a plurality of photodetectors (shown as square, cross-hatched elements in FIG. 4) arranged in a two-dimensional pattern. The pattern enables the detection of light intensity values to be measured at each photodetector, corresponding to multiple separation distances and orientations to the different discrete light sources, each light source corresponding to a plurality of wavelengths. In exemplary embodiments, each light source can generate at least three different wavelengths of light (e.g., 730 nm, 800 nm, and 850 nm).

Light intensity measured at every source-detector pair can be used to calculate the StO₂ parameter in an area of tissue between the light source 102 and photodetector 104. The plurality of StO₂ parameters for a plurality of distinct source-detector pairs can be used to generate a map of StO₂ parameters (i.e., a parameter map) representing tissue oxygen saturation levels at different locations in the tissue.

When using a device with multiple light sources 102, one technique for conducting a measurement can be to turn on each light source 102 in sequence, with only one light source being turned on at a time. Measurements corresponding to a particular light source can be taken by all of the photodetectors 104 simultaneously, each measurement for a particular photodetector corresponding to a particular location and/or separation distance.

In yet other embodiments, the optical device 400 can include light sources and/or photodetectors or other sensors on two or more surfaces of the substrate. Thus, the two or more light sources and two or more photodetectors can be arranged on the substrate in a three-dimensional pattern. Alternatively, the substrate can be arranged with a complex surface, such as a curved surface, such that each of the light sources or photodetectors is located at a position in space indicated by three dimensions, and the separation distance between a light source and the photodetector is the shortest path between the two point in 3D space.

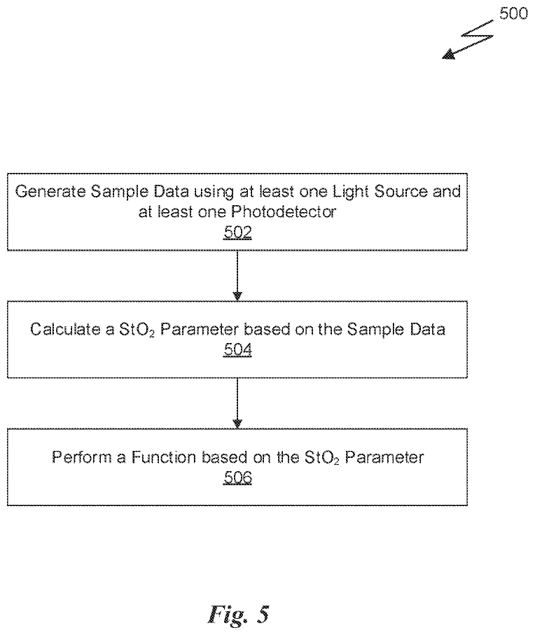

FIG. 5 is a flowchart of a method 500 for measuring a parameter using the device 100, in accordance with some embodiments. Although the method is described in conjunction with the optical device 100, it will be appreciated that the method can be performed using device 150 or device 400 instead of device 100. Furthermore, the steps described herein may be performed, at least in part, using one or more processors, such as the local logic 112 and/or a processor (e.g., CPU, GPU, etc.) included in the mobile device 202 and/or remote computing device 204. The steps may be implemented by executing instructions in any combination of hardware, firmware, and/or software.

At 502, the device 100 generates sample data by measuring light intensity values using the light source(s) 102 and photodetector(s) 104. In an embodiment, the logic 112 is used to activate one or more wavelengths of light of each light source 102 for a period of time. When each light source 102 is activated, the photodetector(s) 104 may be sampled one or more times to measure an intensity of light corresponding to one or more wavelengths that reach the corresponding photodetector site. The sample data can be stored in a memory or streamed to the mobile device 202 and/or remote computing device 204.

At 504, a parameter value is calculated based on the measured sample data. In an embodiment, a StO₂ parameter is calculated based on the light intensity values sampled by each photodetector 104. The equation to calculate the StO₂ parameter can be derived from a modified Beer-Lambert's law, given as Equation 1:

$$Attenuation ( OD ) = - log ( I / {I}_{0} ) = D P F \cdot {μ}_{a} \cdot {d}_{p} + G , \qquad ( Eq . 1 )$$

where I is the light intensity detected at the photodetector 104, I₀ is the light intensity emitted by the light source 102, DPF is the differential pathlength factor, μₐ is the absorption coefficient of the interested tissue, dₚ is the source-detector separation distance, and G is an unknown geometry dependent factor. The unknown geometry dependent factor G makes it impossible to calculate the absolute concentration of hemoglobin. However, this law is often applied to compute the changes in oxy-hemoglobin and deoxy-hemoglobin concentration in a tissue. Since tissue oxygen saturation (the StO₂ parameter) represents a relative concentration of oxy-hemoglobin and deoxy-hemoglobin, this equation can be used as the basis for calculating the StO₂ parameter using light intensity measurements from at least three different wavelengths.

At a specific wavelength, Eq. 1 for a tissue which contains oxy- and deoxy-hemoglobin (HbO, Hb) becomes:

$$O D ( {λ}_{1} ) = {d}_{p} \cdot D P {F}_{1} \cdot ( {ε}_{{λ}_{1}}^{HbO} \cdot {c}_{HbO} + {ε}_{{λ}_{1}}^{Hb} \cdot {c}_{Hb} ) + {G}_{1} , \qquad ( Eq . 2 )$$

where OD is the attenuation,

$${ε}_{{λ}_{1}}^{HbO} and {ε}_{{λ}_{1}}^{Hb}$$

are the extinction coefficients of HbO and Hb, respectively, at wavelength λ₁, and C_{HbO} and C_{Hb} are the concentration of HbO and Hb, respectively. The DPF₁ parameter can be identified by the literature and/or measured by and/or derived from any other technology and/or equations and/or algorithms.

By replacing the concentration with a proportion, Eq. 2 becomes:

$$OD ( {λ}_{1} ) = {d}_{p} \cdot D P {F}_{1} \cdot ( {ε}_{{λ}_{1}}^{HbO} \cdot {p}_{HbO} \cdot {c}_{total} + {ε}_{{λ}_{1}}^{Hb} \cdot {p}_{Hb} \cdot {c}_{total} ) + {G}_{1} , \qquad ( Eq . 3 )$$

where P_{HbO} and P_{Hb} are the proportion of HbO and Hb, respectively, Cₜₒₜₐₗ is the total concentration of hemoglobin, and C_{HbO}=P_{Hbo}·Cₜₒₜₐₗ and C_{Hb}=P_{Hb}·Cₜₒₜₐₗ.

Similarly, Eq. 3 can be replicated at a second wavelength λ₂:

$$OD ( {λ}_{2} ) = {d}_{p} \cdot {DPF}_{2} \cdot ( {ε}_{{λ}_{2}}^{HbO} \cdot {p}_{HbO} \cdot {c}_{total} + {ε}_{{λ}_{2}}^{Hb} \cdot {p}_{Hb} \cdot {c}_{total} ) + {G}_{2} \qquad ( Eq . 4 )$$

By assuming that the geometry dependent factor is the same for the two wavelengths (e.g., G₁=G₂), Eq. 4 can be subtracted from Eq. 3 to get:

$$OD ( {λ}_{1} ) = OD ( {λ}_{2} ) = {d}_{p} \cdot {c}_{total} \cdot \{ {DPF}_{1} \cdot ( {ε}_{{λ}_{1}}^{HbO} \cdot {p}_{HbO} + {ε}_{{λ}_{1}}^{Hb} \cdot {p}_{Hb} ) - {DPF}_{2} \cdot ( {ε}_{{λ}_{2}}^{HbO} \cdot {p}_{HbO} + {ε}_{{λ}_{2}}^{Hb} \cdot {p}_{Hb} ) \} \qquad ( Eq . 5 )$$

Eq. 3 can be replicated again at a third wavelength λ₃:

$$OD ( {λ}_{3} ) - OD ( {λ}_{2} ) = {d}_{p} \cdot {c}_{total} \cdot \{ {DPF}_{3} \cdot ( {ε}_{{λ}_{3}}^{HbO} \cdot {p}_{HbO} + {ε}_{{λ}_{3}}^{Hb} \cdot {p}_{Hb} ) - {DPF}_{2} \cdot ( {ε}_{{λ}_{2}}^{HbO} \cdot {p}_{HbO} + {ε}_{{λ}_{2}}^{Hb} \cdot {p}_{Hb} ) \} \qquad ( Eq . 6 )$$

The ratio of Eq. 5 to Eq. 6 is then given as:

$$\frac{OD ( {λ}_{1} ) - OD ( {λ}_{2} )}{OD ( {λ}_{3} ) - OD ( {λ}_{2} )} = \frac{\begin{matrix}{DPF}_{1} \cdot ( {ε}_{{λ}_{1}}^{HbO} \cdot {p}_{HbO} + {ε}_{{λ}_{1}}^{Hb} \cdot {p}_{Hb} ) - \\ {DPF}_{2} \cdot ( {ε}_{{λ}_{2}}^{HbO} \cdot {p}_{HbO} + {ε}_{{λ}_{2}}^{Hb} \cdot {p}_{Hb} )\end{matrix}}{\begin{matrix}{DPF}_{3} \cdot ( {ε}_{{λ}_{3}}^{HbO} \cdot {p}_{HbO} + {ε}_{{λ}_{3}}^{Hb} \cdot {p}_{Hb} ) - \\ {DPF}_{2} \cdot ( {ε}_{{λ}_{2}}^{HbO} \cdot {p}_{HbO} + {ε}_{{λ}_{2}}^{Hb} \cdot {p}_{Hb} )\end{matrix}} \qquad ( Eq . 7 )$$

Given the property that

$$OD = - log ( I / {I}_{0} ) = log ( {I}_{0} / I ) ,$$

the difference in the optical intensities at two wavelengths can be calculated in terms of the reflected intensities at these wavelengths as:

$$OD ( {λ}_{1} ) - OD ( {λ}_{2} ) = log ( {I}_{{λ}_{1}}^{0} / {I}_{{λ}_{1}} ) - log ( {I}_{{λ}_{2}}^{0} / {I}_{{λ}_{2}} ) = log ( {k}_{1} \cdot {I}_{{λ}_{2}} / {I}_{{λ}_{1}} ) , \qquad ( Eq . 8 )$$

where

$${I}_{{λ}_{1}}^{0} = {k}_{1} \cdot {I}_{{λ}_{2}}^{0} .$$

In other words, kᵢ is a ratio of the intensity of light emitted by the light source 102 for the first wavelength and the second wavelength. In the case where the intensity of light emitted for the first and second wavelengths is the same, k₁=1. Similarly:

$$OD ( {λ}_{3} ) - OD ( {λ}_{2} ) = log ( {I}_{{λ}_{3}}^{0} / {I}_{{λ}_{3}} ) - log ( {I}_{{λ}_{2}}^{0} / {I}_{{λ}_{2}} ) = log ( {k}_{2} \cdot {I}_{{λ}_{2}} / {I}_{{λ}_{3}} ) , \qquad ( Eq . 9 )$$

where

$${I}_{{λ}_{3}}^{0} = {k}_{2} \cdot {I}_{{λ}_{2}}^{0} .$$

In other words, k₂ is a ratio of the intensity of light emitted by the light source 102 for the third wavelength and the second wavelength. In the case where the intensity of light emitted for the third and second wavelengths is the same, k₂=1.

Thus, Eq. 7 becomes:

$$\frac{log ( {k}_{1} \cdot {I}_{{λ}_{2}} / {I}_{{λ}_{1}} )}{log ( {k}_{2} \cdot {I}_{{λ}_{2}} / {I}_{{λ}_{3}} )} = \frac{\begin{matrix}{DPF}_{1} \cdot ( {ε}_{{λ}_{1}}^{HbO} \cdot {p}_{HbO} + {ε}_{{λ}_{1}}^{Hb} \cdot {p}_{Hb} ) - \\ {DPF}_{2} \cdot ( {ε}_{{λ}_{2}}^{HbO} \cdot {p}_{HbO} + {ε}_{{λ}_{2}}^{Hb} \cdot {p}_{Hb} )\end{matrix}}{\begin{matrix}{DPF}_{3} \cdot ( {ε}_{{λ}_{3}}^{HbO} \cdot {p}_{HbO} + {ε}_{{λ}_{3}}^{Hb} \cdot {p}_{Hb} ) - \\ {DPF}_{2} \cdot ( {ε}_{{λ}_{2}}^{HbO} \cdot {p}_{HbO} + {ε}_{{λ}_{2}}^{Hb} \cdot {p}_{Hb} )\end{matrix}} \qquad ( Eq . 10 )$$

In the above, it is assumed that hemoglobin in a tissue mostly exists in either oxy-hemoglobin or deoxy-hemoglobin forms, which means that P_{HbO}+P_{Hb}=1. This assumption is generally true in a normal and healthy individual. However, in patients with methemoglobinemia, there will be elevated met-hemoglobin in the patient's blood. In this case, met-hemoglobin should be taken into account as well. On the other hand,

$${StO}_{2} = \frac{{p}_{HbO}}{{p}_{HbO} + {p}_{Hb}} = {p}_{HbO} .$$

Hence, by substituting StO₂ for P_{HbO} in Eq. 10 and rearranging, we can get an equation for calculating the parameter StO₂ given as:

$${StO}_{2} = \frac{\begin{matrix}{DPF}_{3} \cdot log ( {k}_{2} \cdot {I}_{{λ}_{2}} / {I}_{{λ}_{1}} ) \cdot {ε}_{{λ}_{2}}^{Hb} - {DPF}_{1} \cdot log ( {k}_{2} \cdot {I}_{{λ}_{2}} / {I}_{{λ}_{3}} ) \cdot \\ {ε}_{{λ}_{1}}^{Hb} + {DPF}_{2} \cdot log ( {k}_{2} \cdot {I}_{{λ}_{1}} / {k}_{1} \cdot {I}_{{λ}_{3}} ) \cdot {ε}_{{λ}_{2}}^{Hb}\end{matrix}}{\begin{matrix}{DPF}_{1} \cdot log ( {k}_{2} \cdot {I}_{{λ}_{2}} / {I}_{{λ}_{3}} ) \cdot ( {ε}_{{λ}_{1}}^{HbO} - {ε}_{{λ}_{1}}^{Hb} ) - {DPF}_{3} \cdot log ( {k}_{2} \cdot {I}_{{λ}_{2}} / {I}_{{λ}_{1}} ) \\ ( {ε}_{{λ}_{3}}^{HbO} - {ε}_{{λ}_{3}}^{Hb} ) + {DPF}_{2} \cdot log ( {k}_{1} \cdot {I}_{{λ}_{3}} / {k}_{2} \cdot {I}_{{λ}_{1}} ) ( {ε}_{{λ}_{2}}^{HbO} - {ε}_{{λ}_{2}}^{Hb} )\end{matrix}} \qquad ( Eq . 11 )$$

Table I below provides extinction coefficients for oxy-hemoglobin and deoxy-hemoglobin at three different wavelengths

TABLE I

| | 730 nm | 800 nm | 850 nm |
|---|---|---|---|
| Hemoglobin | (cm⁻¹/moles/L) | (cm⁻¹/moles/L) | (cm⁻¹/moles/L) |
| HbO | 510 | 863.3 | 1097 |
| Hb | 1296.5 | 838.7 | 781 |

The DPF for a particular wavelength of light can be calculated as follows:

$${DPF}_{i} = \frac{1}{2} {( \frac{3 {μ}_{s}^{′} ( {λ}_{i} )}{{μ}_{a} ( {λ}_{i} )} )}^{\frac{1}{2}} , \qquad ( Eq . 12 )$$

where

$${μ}_{s}^{′} = 1 {mm}^{- 1}$$

and μₐ is an absorption coefficient. Example absorption coefficients for 730 nm, 800 nm, and 850 nm for different oxygenation levels of tissue are given below in Table II.

TABLE II

| Oxygenation Level | 730 nm | 800 nm | 850 nm |
|---|---|---|---|
| (%) | (mm⁻¹) | (mm⁻¹) | (mm⁻¹) |
| 0 | 0.029244 | 0.018918 | 0.017617 |
| 10 | 0.02747 | 0.018974 | 0.018329 |
| 20 | 0.025696 | 0.019029 | 0.019042 |
| 30 | 0.023922 | 0.019085 | 0.019755 |
| 40 | 0.022148 | 0.01914 | 0.020468 |
| 50 | 0.020374 | 0.019195 | 0.02118 |
| 60 | 0.0186 | 0.019251 | 0.021893 |
| 70 | 0.016826 | 0.019306 | 0.022606 |
| 80 | 0.015052 | 0.019362 | 0.023319 |
| 90 | 0.013278 | 0.019417 | 0.024032 |
| 100 | 0.011504 | 0.019473 | 0.024744 |

In an embodiment, the μₐ and

$${μ}_{s}^{′}$$

parameters used to calculate the DPFᵢ in Eq. 12 can be any value and can be taken from literature, and/or measured by and/or derived from any other technology and/or equations and/or algorithms.

At 506, the StO₂ parameters are utilized to perform a function. In an embodiment, the function includes setting an alert. The alert can be an audible or visual alert, such as by activating a speaker, buzzer, or other audio output device, displaying a user interface element such as a text box element or a dialog box on a display device, or turning on a light emitting diode on the optical device 100. In an embodiment, the alert can be set in response to determining that the StO₂ parameter is below a threshold value, which can be an indication of hypoxia.

In other embodiments, the function includes transmitting a message to a server computer. The server computer may be included in a network of a healthcare provider. The message can alert a service of a possible hypoxia condition in a patient. The healthcare provider can then take remedial steps to treat the condition. For example, an alarm at a nurse's station can be triggered by the message to alert a nurse that a patient may need urgent care when the StO₂ parameter is below the threshold value.

In yet other embodiments, the function can include processing one or more StO₂ parameters by a machine learning model. A machine learning model can be trained to process an input vector comprising a time-series set of StO₂ parameters collected by the device 100. The input vector can also include other biological parameters related to the health of a user, such as, but not limited to, a relative change in oxy-hemoglobin or deoxy-hemoglobin, a blood oxygen saturation (SpO₂), heart rate and/or heart rate variability, breathing rate and/or breathing depth, temperature, patient movement, or the like. The machine learning model can comprise a neural network, such as a convolutional neural network (CNN), recurrent neural network (RNN), ensemble classifier, or the like. In an embodiment, the neural network can be trained to predict a health condition based on, at least in part, the parameters collected by the optical device 100.

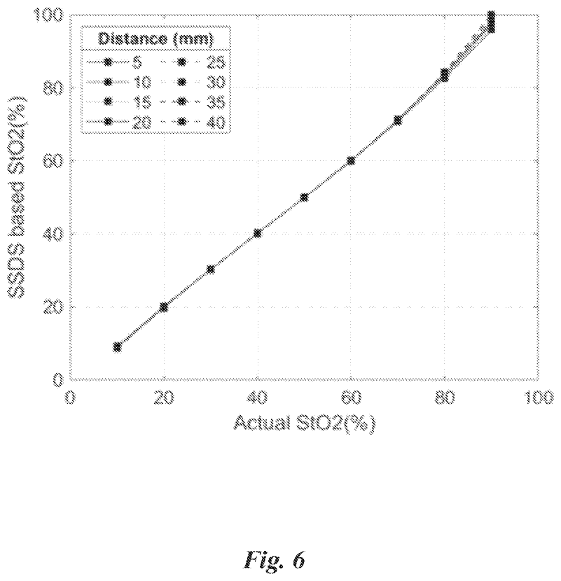

FIG. 6 illustrates a comparison between actual and calculated StO₂ parameters in an optical simulation when DPF values are specified, in accordance with some embodiments. In order to test the theory behind the SSDS-based calculation, a Monte-Carlo optical simulation was conducted. In the simulation, the DPF for three different wavelengths of light (e.g., 730 nm, 800 nm, and 850 nm) is assumed to be the same. Simulated reflectance intensities from each source-detector separation distance between 5 and 40 mm were used to calculate the StO₂ parameter according to Eq. 11. As shown in FIG. 6, the actual StO₂ parameter and the StO₂ parameter calculated using the SSDS-based method are equivalent.

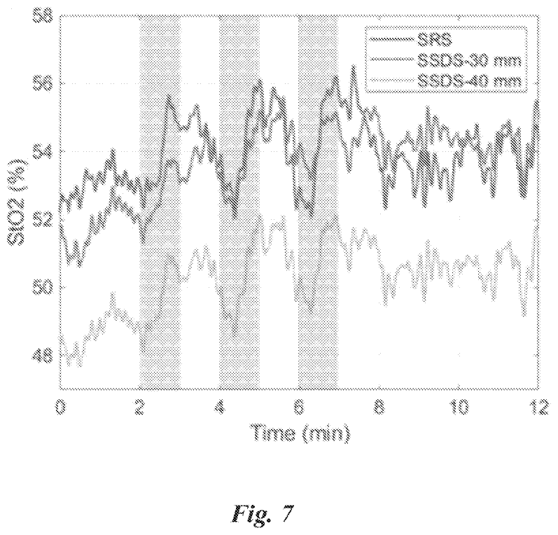

FIG. 7 illustrates a cerebral oxygen saturation calculated using a conventional spatially resolved spectroscopy (SRS) method and the proposed single source-detector separation (SSDS) method, in accordance with some embodiments. The conventional SRS-based method requires at least two distinct separation distances in order to calculate the StO₂ parameter. In comparison, the SSDS-based method described herein can calculate the StO₂ parameter using only a single source-detector pair with a single separation distance between them. The cerebral oxygen saturation level was captured in the pre-frontal cortex during a simulated hypercapnia test in a representative participant in a study. Because the SSDS method needs only one source-detector separation distance to calculate the oxygen saturation level parameter, two separate value sets from two separation distances (e.g., 30 mm and 40 mm) were derived. The StO₂ parameter calculated from both the SRS and the SSDS methods follows the same response trend with a sharp increase during the intervention and a quick return to baseline during a resting state. In comparison to the StO₂ parameter calculated from the 40 mm SSDS, the StO₂ parameter calculated from the 30 mm SSDS appears to be closer to the value calculated using the conventional SRS method.

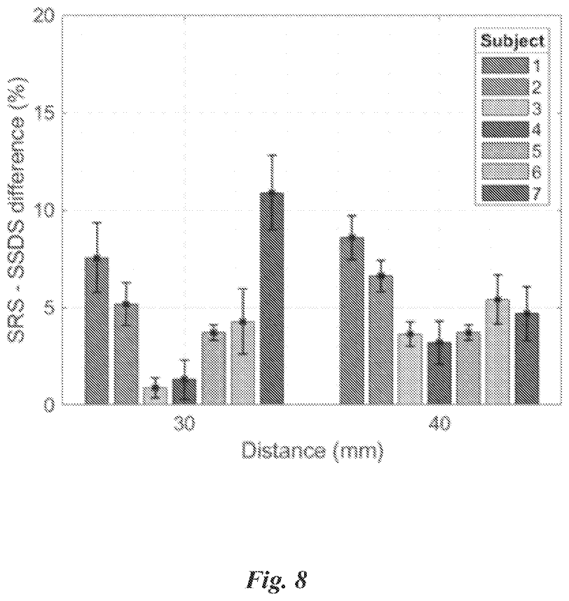

FIG. 8 illustrates a difference in calculated StO₂ parameters compared between the SRS and the SSDS methods, in accordance with some embodiments. A saturation difference can be calculated by taking the absolute difference between the SRS-based StO₂ parameter values and the SSDS-based StO₂ parameter values at each time point. The saturation difference between SRS-based StO₂ parameter values and 30 or 40 mm SSDS-based StO₂ parameter values are relatively small, with average differences of 4.8%±1.2% and 5.1%±1.0%, respectively.

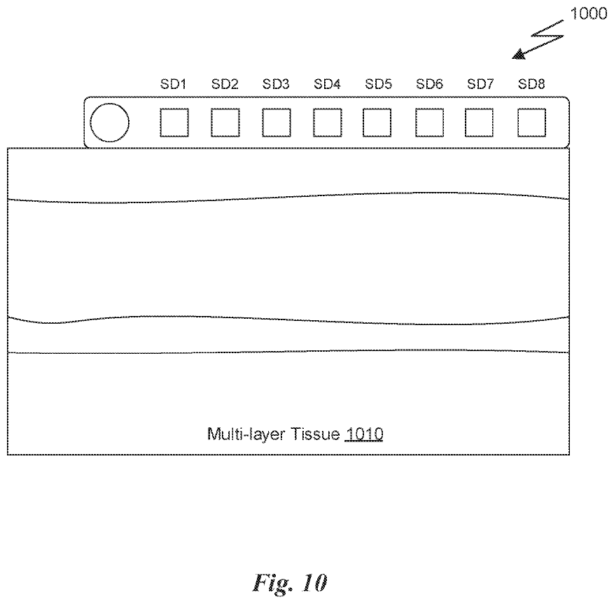

FIG. 10 illustrates an example use case of an optical device, in accordance with some embodiments. As shown in FIG. 10, the optical device 1000 includes one light source and eight photodetectors arranged linearly, with each of the photodetectors labeled as SD1 to SD8, respectively, from smallest to largest separation distance. The signal for SD1 to SD8 will measure tissue oxygen saturation levels at different depths in the multi-layer tissue 1010. In this example, the multi-layer tissue has lower tissue oxygen saturation levels in the first layer (e.g., closest to the device 1000) and higher tissue oxygen saturation levels in the other layers.

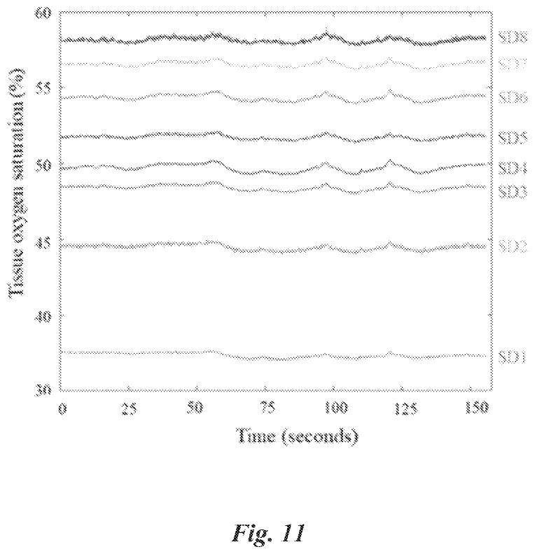

FIG. 11 shows the time-series data sampled using device 1000, in accordance with some embodiments. As shown in FIG. 11, the tissue oxygen saturation level is approximately 35% for SD1, rising to 45% for SD2, and increasing to just under 60% for the deepest layer given for SD8.

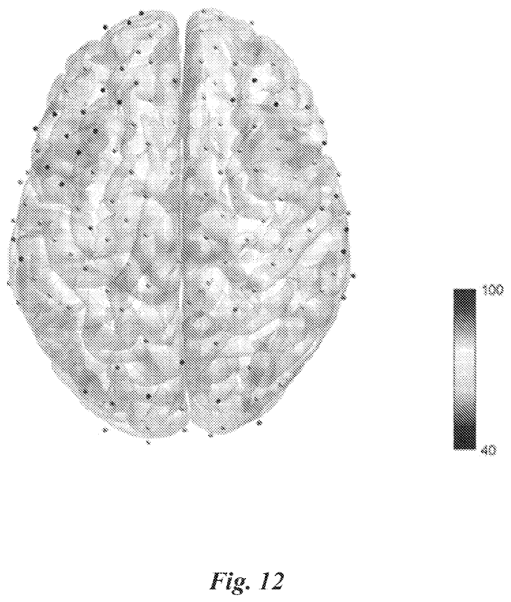

FIG. 12 illustrates a map of cerebral tissue oxygen saturation levels, in accordance with some embodiments. An optical device including a number of light sources and a number of photodetectors can be placed proximate a patient's head. The locations of each source-detector pair can be used to measure a tissue oxygen saturation level in a particular location of the head of the patient, and the different StO₂ parameters from all combinations of source-detector pairs can be used to establish the StO₂ parameter map shown in FIG. 12. A typical CW-NIRS device can only measure a change in cerebral hemodynamics caused by a stimulus. However, the techniques disclosed above can be used to measure brain activity of a patient while resting.

An example system suitable for use in implementing some embodiments of the present disclosure is set forth below. It should be understood that this and other arrangements described herein are set forth only as examples. Other arrangements and elements (e.g., machines, interfaces, functions, orders, groupings of functions, etc.) may be used in addition to or instead of those shown, and some elements may be omitted altogether. Further, many of the elements described herein are functional entities that may be implemented as discrete or distributed components or in conjunction with other components, and in any suitable combination and location. Various functions described herein as being performed by entities may be carried out by hardware, firmware, and/or software. For instance, various functions may be carried out by a processor executing instructions stored in memory. Furthermore, persons of ordinary skill in the art will understand that any system that performs the operations of the method is within the scope and spirit of embodiments of the present disclosure.

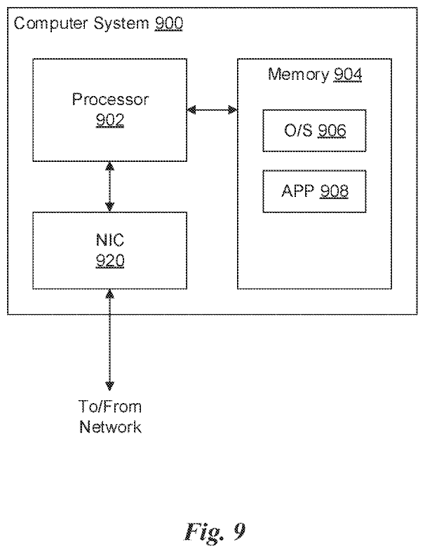

FIG. 9 illustrates an exemplary computer system 900, in accordance with some embodiments. The computer system 900 includes a processor 902, a non-volatile memory 904, and a network interface controller (NIC) 920. The processor 902 can execute instructions that cause the computer system 900 to implement the functionality various elements of the system 200 described above. For example, the mobile device 202 and/or the remote computing device 204 can each take the form of the computer system 900.

Each of the components 902, 904, and 920 can be interconnected, for example, using a system bus to enable communications between the components. The processor 902 is capable of processing instructions for execution within the system 900. The processor 902 can be a single-threaded processor, a multi-threaded processor, a vector processor or parallel processor that implements a single-instruction, multiple data (SIMD) architecture, or the like. The processor 902 is capable of processing instructions stored in the volatile memory 904. In some embodiments, the volatile memory 904 is a dynamic random access memory (DRAM). The instructions can be loaded into the volatile memory 904 from a non-volatile storage, such as a Hard Disk Drive (HDD) or a solid state drive (not explicitly shown), or received via the network. In an embodiment, the volatile memory 904 can include instructions for an operating system 906 as well as one or more applications 908. It will be appreciated that the application(s) can be configured to provide the functionality of one or more components of the system 200, as described above. The NIC 920 enables the computer system 900 to communicate with other devices over a network, including a local area network (LAN) or a wide area network (WAN) such as the Internet.

It will be appreciated that the computer system 900 is merely one exemplary computer architecture and that the processing devices implemented in the system 200 can include various modifications such as additional components in lieu of or in addition to the components shown in FIG. 9. For example, in some embodiments, the computer system 900 can be implemented as a system-on-chip (SoC) that includes a primary integrated circuit die containing one or more CPU cores, one or more GPU cores, a memory management unit, analog domain logic and the like coupled to a volatile memory such as one or more SDRAM integrated circuit dies stacked on top of the primary integrated circuit dies and connected via wire bonds, micro ball arrays, and the like in a single package (e.g., chip). In another embodiment, the computer system 900 can include a printed circuit board with a number of components soldered thereto, as well as one or more expansion cards coupled to an interface such as a peripheral component interconnect (PCI) express (PCIe), or the like. In yet another embodiment, the computer system 900 can be implemented as a server device, which can, in some embodiments, execute a hypervisor and one or more virtual machines that share the hardware resources of the server device.

It is noted that the techniques described herein may be embodied in executable instructions stored in a computer readable medium for use by or in connection with a processor-based instruction execution machine, system, apparatus, or device. It will be appreciated by those skilled in the art that, for some embodiments, various types of computer-readable media can be included for storing data. As used herein, a “computer-readable medium” includes one or more of any suitable media for storing the executable instructions of a computer program such that the instruction execution machine, system, apparatus, or device may read (or fetch) the instructions from the computer-readable medium and execute the instructions for carrying out the described embodiments. Suitable storage formats include one or more of an electronic, magnetic, optical, and electromagnetic format. A non-exhaustive list of conventional exemplary computer-readable medium includes: a portable computer diskette; a random-access memory (RAM); a read-only memory (ROM); an erasable programmable read only memory (EPROM); a flash memory device; and optical storage devices, including a portable compact disc (CD), a portable digital video disc (DVD), and the like.

It should be understood that the arrangement of components illustrated in the attached Figures are for illustrative purposes and that other arrangements are possible. For example, one or more of the elements described herein may be realized, in whole or in part, as an electronic hardware component. Other elements may be implemented in software, hardware, or a combination of software and hardware. Moreover, some or all of these other elements may be combined, some may be omitted altogether, and additional components may be added while still achieving the functionality described herein. Thus, the subject matter described herein may be embodied in many different variations, and all such variations are contemplated to be within the scope of the claims.

To facilitate an understanding of the subject matter described herein, many aspects are described in terms of sequences of actions. It will be recognized by those skilled in the art that the various actions may be performed by specialized circuits or circuitry, by program instructions being executed by one or more processors, or by a combination of both. The description herein of any sequence of actions is not intended to imply that the specific order described for performing that sequence must be followed. All methods described herein may be performed in any suitable order unless otherwise indicated herein or otherwise clearly contradicted by context.

The use of the terms “a” and “an” and “the” and similar references in the context of describing the subject matter (particularly in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. Furthermore, the foregoing description is for the purpose of illustration only, and not for the purpose of limitation, as the scope of protection sought is defined by the claims as set forth hereinafter together with any equivalents thereof. The use of any and all examples, or exemplary language (e.g., “such as”) provided herein, is intended merely to better illustrate the subject matter and does not pose a limitation on the scope of the subject matter unless otherwise claimed. The use of the term “based on” and other like phrases indicating a condition for bringing about a result, both in the claims and in the written description, is not intended to foreclose any other conditions that bring about that result. No language in the specification should be construed as indicating any non-claimed element as essential to the practice of the invention as claimed.

1. An optical device for calculating a tissue oxygen saturation parameter, comprising:
a first light source that generates light having at least three different wavelengths; and
a first photodetector, separated from the light source by a first separation distance, to sample an intensity of light received from the light source at the first photodetector for each of the at least three different wavelengths,
wherein the intensity of light for each of the at least three different wavelengths is used to calculate a first parameter that indicates a tissue oxygen saturation level in one or more layers of tissue between the first light source and the first photodetector, and wherein the first parameter is calculated without the input of any intensity of light measured by any photodetector located at a different separation distance from the light source.

2. The optical device of claim 1, further comprising:
a flexible substrate; and
logic comprising at least a transceiver connected to an antenna to implement a wireless interface.

3. The optical device of claim 2, wherein the logic is configured to transmit sample data to at least one of a mobile device or a remote computing device to calculate the first parameter.

4. The optical device of claim 2, wherein the logic further comprises a processor and a memory, and the processor calculates the first parameter locally and transmits the first parameter to at least one of a mobile device or a remote computing device.

5. The optical device of claim 2, further comprising at least one of:
an inertial measurement unit (IMU) to capture motion data; or
a temperature sensor to capture temperature data.

6. The optical device of claim 5, wherein at least one of the motion data or the temperature data are transmitted to at least one of a mobile device or a remote computing device via the wireless interface.

7. The optical device of claim 1, wherein the first parameter is calculated according to the following equation:
$${StO}_{2} = \frac{\begin{matrix}D P {F}_{3} \cdot log ( {k}_{1} \cdot {I}_{{λ}_{2}} / {I}_{{λ}_{1}} ) \cdot {ε}_{{λ}_{2}}^{Hb} - D P {F}_{1} \cdot log ( {k}_{2} \cdot {I}_{{λ}_{2}} / {I}_{{λ}_{3}} ) \cdot \\ {ε}_{{λ}_{1}}^{Hb} + D P {F}_{2} \cdot log ( {k}_{2} \cdot {I}_{{λ}_{1}} / {k}_{1} \cdot {I}_{{λ}_{3}} ) \cdot {ε}_{{λ}_{2}}^{Hb}\end{matrix}}{\begin{matrix}D P {F}_{1} \cdot log ( {k}_{2} \cdot {I}_{{λ}_{2}} / {I}_{{λ}_{3}} ) ( {ε}_{{λ}_{1}}^{HbO} - {ε}_{{λ}_{1}}^{Hb} ) - D P {F}_{3} \cdot log ( {k}_{1} \cdot {I}_{{λ}_{2}} / {I}_{{λ}_{1}} ) \\ ( {ε}_{{λ}_{3}}^{HbO} - {ε}_{{λ}_{3}}^{Hb} ) + D P {F}_{2} \cdot log ( {k}_{1} \cdot {I}_{{λ}_{3}} / {k}_{2} \cdot {I}_{{λ}_{1}} ) ( {ε}_{{λ}_{2}}^{HbO} - {ε}_{{λ}_{2}}^{Hb} )\end{matrix}} ,$$
where DPFᵢ is a differential pathlength factor, kᵢ is a ratio of intensity of light emitted by the light source for two wavelengths, I_{λi} is the measured intensity of light at the photodetector,
$${ε}_{{λ}_{i}}^{Hb}$$
is the extinction coefficient for deoxy-hemoglobin,
$${ε}_{{λ}_{i}}^{HbO}$$
is the extinction coefficient for oxy-hemoglobin, and i is an index for the different wavelengths of light.

8. The optical device of claim 7, wherein the DPFᵢ is constant for each of the three wavelengths.

9. The optical device of claim 7, wherein the DPFᵢ is calculated for each wavelength according to the following equation:
$$D P {F}_{i} = \frac{1}{2} {( \frac{3 {μ}_{s}^{′} ( {λ}_{i} )}{{μ}_{a} ( {λ}_{i} )} )}^{1 / 2} ,$$
where
$${μ}_{s}^{′} = 1 {mm}^{- 1}$$
and μₐ is an absorption coefficient.

10. The optical device of claim 1, further comprising:
a second photodetector, separated from the first light source by a second separation distance, to sample an intensity of light received from the first light source at the second photodetector for each of the at least three different wavelengths,
wherein the intensity of light for each of the at least three different wavelengths is used to calculate a second parameter that indicates a tissue oxygen saturation level in one or more layers of tissue between the first light source and the second photodetector, and wherein the second parameter is calculated without the input of any intensity of light measured by the first photodetector located at the first separation distance from the first light source.

11. The optical device of claim 1, further comprising:
one or more additional photodetectors to measure a second parameter, wherein the second parameter indicates at least one of a heart rate or a pulse oxidation.

12. The optical device of claim 1, further comprising:
one or more additional light sources of one or more wavelengths used to measure a second parameter, wherein the second parameter indicates at least one of a heart rate or a pulse oxidation.

13. The optical device of claim 1, further comprising:
one or more additional light sources of one or more wavelengths and one or more additional photodetectors to measure a second parameter, wherein the second parameter indicates at least one of a heart rate or a pulse oxidation.

14. The optical device of claim 13, wherein the one or more additional light sources and one or more additional photodetectors are located on one or more surfaces of a substrate.

15. The optical device of claim 13, wherein the one or more additional light sources and one or more additional photodetectors are arranged in a three-dimensional pattern on two or more surfaces of a substrate.

16. The optical device of claim 1, wherein the first light source comprises multiple light sources with at least one wavelength, wherein each of the multiple light sources are placed close to the other multiple light sources to act as a multi-wavelength light source.

17. The optical device of claim 1, further comprising:
at least one additional light source; and
a plurality of additional photodetectors arranged in a two-dimensional pattern,
wherein, for each additional photodetector of the plurality of additional photodetectors, the intensity of light for each of the at least three different wavelengths is used to calculate a corresponding parameter that indicates a tissue oxygen saturation level in one or more layers of tissue between a particular light source and the additional photodetector, wherein the particular light source is selected U.S. National Stage Application Preliminary Amendment from the first light source or one of the at least one additional light source, and wherein the corresponding parameter is calculated without the input of any intensity of light measured by any of the other additional photodetectors located at a different separation distance from the particular light source, and
wherein a plurality of corresponding parameters for the plurality of additional photodetectors are used to construct a parameter map.

18. (canceled)

19. (canceled)

20. (canceled)

21. A system for determining a tissue oxygen saturation StO₂ parameter of an in-vivo of a subject, the system comprising:
an optical device; and
a remote computing device in communication with the optical device,
wherein at least one of the optical device or the remote computing device is configured to calculate the StO₂ parameter in accordance with a single source-detector separation (SSDS)-based method that is based on light intensity measurements performed by the optical device for at least three wavelengths of light emitted by a first light source and measured at a first photodetector separated from the light source by a first separation distance.

22. (canceled)

23. (canceled)

24. The system of claim 21, wherein the remote computing device is configured to execute a machine learning algorithm based on at least one StO₂ parameter measured by the optical device.

25. (canceled)

26. (canceled)

27. (canceled)

28. A method comprising:
measuring, using a first photodetector of an optical device, an intensity of light for at least three wavelengths of light emitted by a first light source to generate sample data, wherein the light source is positioned a first separation distance from the first photodetector; and
calculating a StO₂ parameter using a single source-detector separation (SSDS)-based method in accordance with the following equation:
$${StO}_{2} = \frac{\begin{matrix}D P {F}_{3} \cdot log ( {k}_{1} \cdot {I}_{{λ}_{2}} / {I}_{{λ}_{1}} ) \cdot {ε}_{{λ}_{2}}^{Hb} - D P {F}_{1} \cdot log ( {k}_{2} \cdot {I}_{{λ}_{2}} / {I}_{{λ}_{3}} ) \cdot \\ {ε}_{{λ}_{1}}^{Hb} + D P {F}_{2} \cdot log ( {k}_{2} \cdot {I}_{{λ}_{1}} / {k}_{1} \cdot {I}_{{λ}_{3}} ) \cdot {ε}_{{λ}_{2}}^{Hb}\end{matrix}}{\begin{matrix}D P {F}_{1} \cdot log ( {k}_{2} \cdot {I}_{{λ}_{2}} / {I}_{{λ}_{3}} ) ( {ε}_{{λ}_{1}}^{HbO} - {ε}_{{λ}_{1}}^{Hb} ) - D P {F}_{3} \cdot log ( {k}_{1} \cdot {I}_{{λ}_{2}} / {I}_{{λ}_{1}} ) \\ ( {ε}_{{λ}_{3}}^{HbO} - {ε}_{{λ}_{3}}^{Hb} ) + D P {F}_{2} \cdot log ( {k}_{1} \cdot {I}_{{λ}_{3}} / {k}_{2} \cdot {I}_{{λ}_{1}} ) ( {ε}_{{λ}_{2}}^{HbO} - {ε}_{{λ}_{2}}^{Hb} )\end{matrix}} ,$$
where DPFᵢ is a differential pathlength factor, kᵢ is a ratio of intensity of light emitted by the light source for two wavelengths, I_{λi} is the measured intensity of light at the photodetector,
$${ε}_{{λ}_{i}}^{Hb}$$
is the extinction coefficient for deoxy-hemoglobin,
$${ε}_{{λ}_{i}}^{HbO}$$
is the extinction coefficient for oxy-hemoglobin, and i is an index for the different wavelengths of light.
